SEQUENCE LISTING (4 sequences)

SEQ ID NO: 1 - DNA, 21 bp, Artificial Sequence
  Synthetic oligonucleotide primer
gcaaaggctattccacagcag

SEQ ID NO: 2 - DNA, 20 bp, Artificial Sequence
  Synthetic oligonucleotide primer
agcatttcagttcccaccac

SEQ ID NO: 3 - DNA, 20 bp, Artificial Sequence
  Synthetic oligonucleotide primer
gcacgccatcactatcttcc

SEQ ID NO: 4 - DNA, 20 bp, Artificial Sequence
  Synthetic oligonucleotide primer
catccaccgtcttctgtgtg

(19) United States
(12) Patent Application Publication
(10) Pub. No.: US 2019/0054057 A1
(43) Pub. Date: Feb. 21, 2019

(54) IMMUNE BOOSTING DIETARY COMPOUNDS FOR DISEASE CONTROL AND PREVENTION

(71) Applicant: THE BOARD OF REGENTS FOR OKLAHOMA STATE UNIVERSITY OFFICE OF INTELLECTUAL PROPERTY MANAGEMENT, Stillwater, OK (US)

(72) Inventor: Guolong Zhang, Stillwater, OK (US)

(21) Appl. No.: 16/079,730

(22) PCT Filed: Mar. 8, 2017
(86) PCT No.: PCT/US17/21392

§ 371 Date: Aug. 24, 2018

Related U.S. Application Data
(60) Provisional application No. 62/305,242, filed on Mar. 8, 2016.

(51) Int. Cl. A61K 31/225; A61K 31/222; A61K 31/216; A61K 31/7076; A23K 20/158; A23K 20/153; A23K 20/111; A23K 50/75

(52) U.S. Cl. CPC A61K 31/225; A61K 31/222; A61K 31/216; A23K 50/75; A23K 20/158; A23K 20/153; A23K 20/111; A61K 31/7076

(57) ABSTRACT

Provided herein are small-molecule compounds and combinations thereof that enhance the synthesis of animal endogenous genus host defense peptides, which display potent antimicrobial and immunomodulatory activities. They thus represent alternatives to antibiotics for disease control and prevention for use in animals and humans. Examples of the small molecule compounds include histone deacetylase inhibitors, mono- and disaccharide sugars, cyclic adenosine monophosphate (cAMP) signaling agonists, and cyclooxygenase -2 (COX-2) inhibitors. Synergistic combinations include short-chain fatty acids, their chemical analogs or histone deacetylase inhibitors with other fatty acids, sugars, cAMP signaling agonists, and/or COX-2 inhibitors.

### CROSS REFERENCE TO RELATED APPLICATIONS

This application claims the benefit of U.S. Provisional Patent Application Ser. No. 62/305,242 filed on Mar. 8, 2016, and incorporates said provisional application by reference into this document as if fully set out at this point.

### GOVERNMENT RIGHTS

This invention was made with United States Government support under USDA/NIFA Grant No. 2008-35204-04544 awarded by the Department of Agriculture. The United States Government has certain rights in this invention.

### TECHNICAL FIELD

This disclosure relates generally to antimicrobial disease control and prevention and, more particularly, to immune boosting dietary compounds for disease control and prevention that are alternatives to traditional antibiotics.

### BACKGROUND

Routine use of antibiotics at subtherapeutic levels in animal feed has been criticized as a major driving force for the emergence of antibiotic-resistant pathogens, a serious public health concern worldwide. Although various alternatives to antibiotics, including direct-fed microbials, botanical extracts, feed enzymes, bacteriophages, bacteriocins, and acidifiers have been explored in the livestock industry, none has been shown to match the cost-effectiveness, convenience, and performance offered by current conventional antibiotics. Unfortunately, most of the immunomodulators currently on the market nonspecifically stimulate a broad spectrum of immune and inflammatory responses, which often adversely affect animal growth and health.

What is urgently needed are compounds that confer on the host the ability to fight off infections without triggering inflammation and without inducing antimicrobial resistance, while maintaining optimal animal health and performance.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

Provided herein are small molecule compounds, and in some embodiments, synergistic combinations of the compounds, that have a strong ability to enhance the synthesis of endogenous host defense peptides (HDPs) in animals. HDPs (also referred to as antimicrobial peptides or “AMPs”) are part of the innate immune response and constitute a group of critical first-line defense molecules with antimicrobial and immunomodulatory activities. These peptides are potent, broad spectrum antibiotics which have been demonstrated to kill Gram negative and Gram positive bacteria, enveloped viruses, fungi and even transformed or cancerous cells. Unlike the majority of conventional antibiotics, HDPs also enhance immunity by functioning as immunomodulators. The compounds synergize strongly with each other to augment HDP synthesis both in cells and in live animals. Administration of the small molecule combinations, e.g. in animal feed or water, causes an increase in endogenous production of HDPs, thereby decreasing or eliminating infectious microbes in the animal. For example, supplementation of feed with the compounds reduced the Salmonella titer in the cecum of chickens following experimental infections.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1A and FIG. 1B. Induction of AvBD9 gene expression by butyrate analogs. Chicken HD11 cells were treated with or without different concentrations of individual structural analogs of butyrate (A) for 24 h, followed by RNA isolation and real-time RT-PCR analysis of AvBD9 expression (B). Each bar represents mean±standard error of the data from two independent experiments.

FIG. 2A and FIG. 2B. Induction of AvBD9 gene expression by histone deacetylase inhibitors. Chicken HD11 cells were treated with or without different concentrations of individual histone deacetylase inhibitors (A) for 24 h, followed by RNA isolation and real-time RT-PCR analysis of AvBD9 expression (B). Each bar represents mean±standard error of the data from two independent experiments.

FIG. 3. Time-dependent induction of AvBD9 gene expression by quercetin. Chicken HTC macrophage cells were incubated with 40 μM quercetin for the indicated times, followed by RNA isolation and real time RT-PCR analysis of AvBD9 gene expression. Data were obtained from 2 or 3 independent experiments. The bars without common superscript letters denote significance (P<0.05 by unpaired Student's t-test).

FIG. 4A, FIG. 4B, and FIG. 4C. Synergistic induction of AvBD9 gene expression by butyrate and quercetin. Chicken HTC macrophage cells (A) and peripheral blood mononuclear cells (PBMCs) (B) were incubated with 2 mM butyrate in the presence or absence of differing concentrations of quercetin for 24 h. Separately, 2 mM butyrate and 10 μM quercetin were used to stimulate PBMCs for the indicated times (C). Cells were then harvested and subjected to total RNA isolation and real-time RT-PCR analysis of the AvBD9 expression. Data was obtained from 2 or 3 independent experiments. The bars without common superscript letters denote significance (P<0.05 by unpaired Student's t-test).

FIG. 5A, FIG. 5B, and FIG. 5C. Effect of natural COX-2 inhibitors on AvBD9 expression in butyrate-stimulated chicken PBMCs. After 1 h pre-incubation with resveratrol (A), anacardic acid (B) or garcinol (C), chicken PBMCs were stimulated with 2 mM butyrate for 24 h, followed by RNA isolation and real time RT-PCR analysis of the AvBD9 expression. Each bar shows mean±standard error of the data from 2 or 3 experiments. The bars without common superscript letters denote significance (P<0.05 by unpaired Student's t-test).

FIG. 6. Synergistic induction of AvBD9 gene expression between butyrate and synthetic COX-2 inhibitors. Chicken PBMCs were incubated with different concentrations of two synthetic inhibitors, nimesulide and niflumic acid, for 1 h followed by 2 mM butyrate for another 24 h. Real time RT-PCR analysis was performed to evaluate AvBD9 gene expression. Data was obtained from three independent experiments. The bars without common superscript letters denote significance (P<0.05 by unpaired Student's t-test).

FIG. 7. Role of MAPK, NF-κB, and cAMP signaling pathways in AvBD9 induction mediated by butyrate and quercetin. Chicken PBMC cells were pretreated for 1 h with or without 25 μM 5B203580 (p38 MAPK inhibitor), 20 μM SP600125 (JNK inhibitor), 50 μM PD98059 (MEK½ inhibitor), 40 μM PDTC (NF-κB inhibitor), 20 μM MG132 (NF-κB inhibitor), and 100 and 500 μM DDA (adenylate cyclase/cAMP signaling inhibitor), followed by stimulation with 2 mM sodium butyrate with or without 10 μM quercetin for another 24 h. Real-time RT-PCR was performed to determine AvBD9 mRNA expression. The bars without common superscript letters denote significance (P<0.05 by unpaired Student's t-test).

FIG. 8A and FIG. 8B. Time- and dose-dependent induction of AvBD9 gene expression by sugars. Chicken HD11 macrophage cells were incubated with 0.2 M of indicated sugars for 3, 6, 12, 24 and 48 h (A), or incubated with 0.1 or 0.2 M of indicated sugars for 6 h (B). Cells were then subjected to total RNA isolation and real-time PCR analysis of the AvBD9 gene expression. Each bar shows mean±standard error of the data from 2 or 3 independent experiments.

FIG. 9A, FIG. 9B, and FIG. 9C. Synergistic induction of AvBD9 gene expression between butyrate and sugars. (A) Chicken HD11 macrophage cells were incubated with 2 mM sodium butyrate or 0.2 M lactose individually or in combination for various times. (B) Chicken jejunal explants were incubated with 2 mM sodium butyrate or 0.1 M lactose individually or in combinations for 24 h. (C) Chicken HD11 cells were incubated for 12 h with 2 mM butyrate or 0.2 M of indicated sugars separately or in combinations. Cells were then subjected to total RNA isolation and real-time PCR analysis of the AvBD9 gene expression. Each bar shows mean±standard error of the data from 2-3 independent experiments.

FIG. 10. Synergistic induction of chicken HDP gene expression between butyrate and sugars. Chicken HD11 macrophage cells were incubated with 2 mM sodium butyrate, 0.2 M lactose, 0.2 M lactose individually or in combination for various times. Cells were then subjected to total RNA isolation and real-time PCR analysis of the gene expression of AvBD1-13. Each bar shows mean±standard error of the data from 2-3 independent experiments.

FIG. 11A and FIG. 11B. Role of histone acetylation in AvBD9 induction by butyrate and dietary compounds. (A) Chicken HD11 cells treated with 2 mM butyrate and/or 0.2 M lactose for 6, 12 or 24 h. (B) Chicken HD11 cells treated with 2 mM butyrate and/or 10 μM quercetin for 6 or 12 h. The impact on the acetylation of histones was revealed by immunoblotting with mAb against acetyl-histone 4 (Ac-H4) (Cell Signaling, #8647). β-actin staining was using to show a similar amount of protein loading in each lane.

FIG. 12. Role of MAPK, NF-κB, and cAMP signaling pathways in AvBD9 induction mediated by butyrate and lactose. Chicken HD11 cells were pretreated for 1 h with or without specific inhibitors: 25 μM SB203580 (MEK½), 20 μM SP600125 (p38 MAPK), 50 μM PD98059 (JNK), 0.1 μM QNZ (NF-κB), and 1 mM SQ22536 (cAMP), followed by stimulation with 2 mM sodium butyrate with or without 0.2 M lactose for another 24 h. Real-time RT-PCR was performed to determine AvBD9 mRNA expression. Each bar shows mean±standard error of the data from 2-3 independent experiments.

FIG. 13A and FIG. 13B. Synergistic induction of AvBD9 gene expression among butyrate, forskolin, and lactose. (A) Chicken HD11 macrophage cells were incubated with 1 mM sodium butyrate, 2.5 μM forskolin or 0.2 M lactose individually or in combinations for 24 h. (B) Chicken jejunal explants were incubated with 2 mM sodium butyrate, 2.5 μM forskolin or 0.2 M lactose individually or in combinations for 24 h. Cells were then subjected to total RNA isolation and real-time PCR analysis of the AvBD9 gene expression. Each bar shows mean±standard error of the data from 2-3 independent experiments.

FIG. 14. Synergistic induction of chicken HDP gene expression between butyrate and forskolin. Chicken HD11 macrophage cells were incubated with 2, 5, 10 or 20 μM forskolin in the presence of absence of 2 mM sodium butyrate for 24 h. Cells were then subjected to total RNA isolation and real-time PCR analysis of gene expressions of chicken HDPs. The color elements represent average log2 ratios of fold change from 2-3 independent experiments. Gray indicates up-regulation, whereas black means no induction.

FIG. 15A and FIG. 15B. Synergistic induction of AvBD9 gene expression between butyrate and medium- or long-chain fatty acids. Chicken HD11 cells were incubated with 2 mM butyrate in the presence or absence of indicated concentrations of a medium-chain fatty acid (sodium octanoate) (A) or three different long-chain fatty acids (CLA, linolenic acid, and linoleic acid) (B) for 24 h. Real time RT-PCR analysis was performed to evaluate AvBD9 gene expression. Data was obtained from 2 or 3 independent experiments.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

Provided herein are synergistic combinations of agents and methods of using the synergistic combinations to increase the expression of genes encoding host defense peptides (HDPs).

By “synergy” or “synergistic” we mean that the interaction of two or more substances to produce a combined effect is greater than the sum of their separate effects.

As used herein, a “structural” or “chemical” analog is a compound having a chemical structure similar to that of another one, but differing from it in respect of certain components, e.g. differing by one or more atoms, functional groups, or substructures, which are replaced with other atoms, groups, or substructures.

As used herein, a “functional” analog is a chemical compound that has properties or activities similar to those of another compound (e.g. physical, chemical, biochemical, or pharmacological properties such as mechanism or target of action), without necessarily sharing structural similarity, i.e. the chemical structures differ.

As used herein, a “fatty acid” is a carboxylic acid with a long aliphatic chain, which is either saturated or unsaturated. Most naturally occurring fatty acids have an unbranched chain (“tail”) of an even number of carbon atoms, from 4 to 28. A “short chain fatty acid” is a fatty acid with less than 6 carbon atoms, i.e. with 5 or fewer carbon atoms, such as 5, 4, 3, 2, or 1 carbon atom(s). A medium-chain fatty acid (MCFA) is a fatty acid with an aliphatic tail of 6-12 carbons, and a long-chain fatty acids (LCFA) is a fatty acid with an aliphatic tail of 13 to 21 carbons. A very long chain fatty acid (VLCFA) is a fatty acid with an aliphatic tail longer than 22 carbons.

As used herein, a monosaccharide (or simple sugar) is a sugar that cannot be hydrolyzed into smaller sugar units. Monosaccharides generally have from about 1 to about 7 carbon atoms, and include, e.g., trioses, tetroses, pentoses, hexoses, and heptoses. The monosaccharides may be linear or cyclic, and stereoisomers are also encompassed.

As used herein, a disaccharide (double sugar, biose) is a sugar formed when two monosaccharides are joined by a glycosidic linkage. Both linear and cyclic forms, and stereoisomers thereof, are encompassed.

According to some aspects, the small molecules used in the practice of the present include the following:

Class I. Fatty Acids and Structural Analogs Thereof.

A1. Short-chain fatty acids (SCFAs) including, but not limited to: acetic (C2), propionic (C3), butyric (C4), isobutyric acid, valeric acid (C5), isovaleric acid, and salts thereof.

A2. Structural analogs of SCFAs including, but not limited to: monoglyceride, diglyceride and triglyceride analogs of SCFAs such as glyceryl tributyrate, glyceryl dibutyrate, and glyceryl monobutyrate; benzyl analogs of SCFAs such as benzyl butyrate, benzyl propionate and benzyl valerate; cinnamyl and trans-cinnamyl analogs of SCFAs such as trans-cinnamylbutyrate; and SCFAs with a phenyl group attached such as hydrocinnamic acid and 4-phenylbutyrate; and salts thereof.

B1. Medium-chain fatty acids (MCFAs) including, but not limited to: caproic (C6), enanthic (C7), caprylic (C8), pelargonic (C9), capric (C10), undecylic (C11), and lauric acid (C12), and their isomers, and salts thereof.

B2. Structural analogs of MCFAs including, but not limited to: monoglyceride, diglyceride and triglyceride analogs of MCFAs, benzyl analogs of MCFAs, cinnamyl and trans-cinnamyl analogs of MCFAs, and MCFAs with a phenyl group attached, and salts thereof.

C1. Saturated and unsaturated long-chain fatty acids (LCFAs) including but not limited to: tridecylic (C13), myristic (C14), pentadecanoic (C15), palmitic (C16), margaric (C17), stearic (C18), nonadecylic (C19), arachidic (C20), heneicosylic (C21), behenic (C22), α-linolenic (18:3), stearidonic (18:4), eicosapentaenoic (20:5), docosahexaenoic (22:6), linoleic (18:2), conjugated linoleic, γ-linolenic (18:3), dihomo-γ-linolenic (20:3), arachidonic (20:4), adrenic (22:4), palmitoleic (16:1), vaccenic (18:1), paullinic (20:1), oleic (18:1), elaidic (trans-18:1), gondoic (20:1), erucic (22:1), nervonic (24:1), and mead acid (20:3), and their isomers, and salts thereof.

C2. Structural analogs of saturated and unsaturated LCFAs including but not limited to: monoglyceride, diglyceride and triglyceride analogs of LCFAs, benzyl analogs of LCFAs, cinnamyl and trans-cinnamyl analogs of LCFAs, and LCFAs with a phenyl group attached, and salts thereof.

Class II. Functional analogs of SCFAs, i.e., histone deacetylase inhibitors including but not limited to: sodium valproate, Vorinostat (SAHA, MK0683), trichistatin A, CAY10433/BML-210, CAY10398, Entinostat (MS-275), Chidamide, Trichostatin A (TSA), Panobinostat (LBH589), Mocetinostat (MGCD0103), Belinostat (PXD101), Romidepsin (FK228, Depsipeptide), MC1568, Tubastatin A-HCl, Tubastatin A, Givinostat (ITF2357), LAQ824 (Dacinostat), CUDC-101, Quisinostat (JNJ-26481585), Pracinostat (SB939), PCI-34051, Droxinostat, PCI-24781 (Abexinostat), RGFP966, AR-42, Rocilinostat (ACY-1215), CI994 (Tacedinaline), CUDC-907, M344, Tubacin, RG2833 (RGFP109), Resminostat, BRD73954, BG45, 4SC-202, CAY10603, LMK-235, Nexturastat A, TMP269, Scriptaid, and HPOB.

Class III. Mono- and disaccharides and their chemical and structural analogs, examples of which include but are not limited to: monosaccharides such as D- and L-isomers of hexoses (allose, altrose, glucose, mannose, gulose, idose, galactose, talose, fructose, psicose, sorbose, and tagatose) and disaccharides such as sucrose, lactose, maltose, trehalose, lactulose, and cellobiose. Chemical and structural analogs of sugars include, for example, sugar alcohols such as mannitol, etc.

Class IV. Agonists of cyclic adenosine monophosphate (cAMP) signaling pathways, examples of which include but are not limited to: 8-bromo-cAMP, forskolin, cholera toxin (CT), pertussis toxin (PT), dibutyryl cAMP (or bucladesine), caffeine, and theophylline.

Class V. Cyclooxygenase-2 inhibitors, examples of which include but are not limited to: quercetin, resveratrol, garcinol, anacardic acid, curcumin, epigallocatechin-3 galate, pycnogenol nimesulide, niflumic acid, celecoxib, etoricoxib, and rofecoxib.

A synergistic effect on host defense peptide gene expression was observed when 2-3 compounds within or between different classes are combined into categories i)-iv), as follows:

Category i): According to some aspects of the invention, the compositions provided herein comprise 2-3 Class I compounds. Exemplary synergistic combinations in Category i) include but are not limited to: a combination of 2-3 SCFAs or structural analogs thereof, and a combination of a SCFA with a MCFA or a LCFA or structural analogs thereof.

Category ii): According to other aspects of the invention, the compositions comprise two or more compounds, at least one of which is a Class I compound and at least one of which is a Class II compound. Exemplary synergistic combinations within category ii) include but are not limited to: a combination of a histone deacetylase inhibitor such as butyrate or SAHA with a SCFA, MCFA or LCFA.

Category iii): According to other aspects of the invention, the compositions comprise two or more compounds, at least one of which is a Class II compound (or Class II-A1/A2 compound) and at least one of which is a Class III mono- or disaccharide sugar. Exemplary synergistic combinations within category iii) include but are not limited to: a combination of a histone deacetylase inhibitor such as butyrate or SAHA with lactose or galactose.

Category iv): According to other aspects of the invention, the compositions comprise two or more compounds, at least one of which is Class II compound (or Class II-A1/A2 compound) and at least one of which is a Class IV cAMP signaling agonist. Exemplary synergistic combinations within category iv) include but are not limited to: a combination of a histone deacetylase inhibitor such as butyrate or SAHA with forskolin or bucladesine.

Category v): According to other aspects of the invention, the compositions comprise two or more compounds, at least one of which is a Class II compound (or Class II-A1/A2 compound) and at least one of which is a Class V cyclooxygenase-2 inhibitor. Exemplary synergistic combinations within category v) include but are not limited to: a combination of a histone deacetylase inhibitor such as butyrate or SAHA with quercetin, resveratrol, anacardic acid or garcinol.

Category vi): According to other aspects of the invention, the compositions comprise three or more compounds, at least one of which is a Class II compound (or Class II-A1/A2 compound) and the remaining two or more of which are from at least two different categories, the categories being selected from Class III, Class IV and Class V. For example, a Class II compound may be combined with one or more compounds from Class III and one or more from Class IV; or one or more compounds from Class III and one or more from Class V; or one or more compounds from Class IV and one or more from Class V. Exemplary synergistic combinations within category vi) include but are not limited to: a combination of a histone deacetylase inhibitor (such as butyrate or SAHA) with a cAMP signaling agonist (such as forskolin) and a sugar (such as lactose).

The agents described herein exhibit synergy with respect to increasing the level of expression of one or more genes encoding an antimicrobial host defense peptide, when administered in the combinations described herein. Generally, the combinations are administered as a single composition, i.e. the composition is a mixture of the two or more agents. However, administration of the agents separately is also encompassed, in which case administration is generally coordinated so that the subject takes in all the agents in the combination at least during a single day or one week period, and usually within a shorter time frame, e.g. within at least 10, 9, 8, 7, 6, 5, 4, 3, 2, or 1 hour(s) or less, so the agents are present together within the subject at the same time, the physiological activity of each is carried out at the same or at least at overlapping times, and the synergistic effect is thus realized. For example, one agent may be present in dry feed and the second agent may be present in drinking water, etc., both of which are consumed throughout the day. Or, if the subject is a human or a non-human animal that can be dosed by hand, the components of a combination may be administered one after the other, e.g. in two separate preparations. For example, one component may require an aqueous carrier and a second and/or third component may require an oil-based carrier, so that they cannot, or cannot easily, be combined into a single preparation. However, it is generally preferred to administer all agents of a combination at once as a single composition.

Accordingly, in some aspects, the compositions described herein comprise at least two of the compounds described herein, i.e. two or more different compounds (such as 2, 3, 4, 5, 6, 7, 8, 9, 10 or more) in a single formulation. Accordingly, the present invention encompasses such formulations/compositions. The two or more compounds are generally substantially purified and are generally present in a pharmacologically suitable (physiologically compatible) carrier, which may be aqueous or oil-based. In some aspects, such compositions are prepared as liquid solutions or suspensions, or as solid forms such as tablets, pills, powders and the like. Solid forms suitable for solution in, or suspension in, liquids prior to administration are also contemplated (e.g. dried, desiccated or lyophilized forms of the combinations), as are emulsified preparations. In some aspects, the liquid formulations are aqueous or oil-based suspensions or solutions. In some aspects, the active ingredients are mixed with excipients which are pharmaceutically acceptable and compatible with the active ingredients, e.g. pharmaceutically acceptable salts. Suitable excipients or carriers include, for example, water, ethanol, saline, DMSO, dextrose, glycerol, starch, limestone, microspheres, nanoparticles and the like, or combinations thereof. In addition, the composition may contain minor amounts of auxiliary substances such as wetting or emulsifying agents (such as vegetable oils, liposomes and polymeric micelles), pH buffering agents, preservatives, and the like. In some aspects, the compounds are crude extracts of certain plants or animals.

Generally, when the subject is a non-human animal, the compositions are mixed with or added to animal feed, either during commercial preparation of the feed, or added to the feed as a supplement prior to providing the feed to the animal. The feed may be solid or liquid, e.g. adult subjects generally eat solid feed but young subjects (e.g. newborns) may need liquid food. Alternatively, the compositions may be added to drinking water, or administered by any other suitable route, e.g. by individual dosing as described above.

If it is desired to administer an oral form of the composition, or to provide a form that is suitable for mixing with animal feed, various thickeners, flavorings, diluents, emulsifiers, dispersing aids, bulking agents or binders and the like may be added. The composition may contain any such additional ingredients so as to provide the composition in a form suitable for administration, either directly or via a food product. The final amount of compound in the formulations varies, but is generally from about 1-99%. Still other suitable formulations for use in the present invention are found, for example in Remington's Science and Practice of Pharmacy, 22nd ed. (Pharmaceutical Press 2012, ISBN: 0857110624).

Some examples of materials which can serve as pharmaceutically acceptable carriers include, but are not limited to, ion exchangers, alumina, aluminum stearate, lecithin, serum proteins (such as human serum albumin), buffer substances (such as Tween® 80, phosphates, glycine, sorbic acid, or potassium sorbate), vegetable oils, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The compositions may be administered in vivo by any suitable route including but not limited to: by inclusion in a food product, by inoculation or injection (e.g. intravenous, intraperitoneal, intramuscular, subcutaneous, and the like), by topical application and by absorption through epithelial or mucocutaneous linings. Other suitable means include but are not limited to: inhalation (e.g. as a mist or spray), orally (e.g. as a pill, capsule, liquid, etc.), intravaginally, intranasally, rectally, by ingestion of a food or probiotic product containing the combinations of agents, as eye drops, incorporated into dressings or bandages (e.g. lyophilized forms may be included directly in the dressing), etc. In some aspects, for non-human animals administration is via a food product or drinking water. For a human subject, the mode of administration is typically oral or by injection. In addition, the compositions may be administered in conjunction with other treatment modalities such as substances that boost the immune system, various chemotherapeutic agents, one or more antibiotic agents (e.g. conventional antibiotics, but generally at a lower dose than when administered without the combinations described herein), vitamins, minerals, and the like.

Suitable subjects which would benefit by administration of the compositions described herein include but are not limited to various vertebrate animals such as fishes, amphibians, reptiles, birds and mammals, including both human and non-human mammals. The fishes, birds and non-human mammals include those which are raised for commercial purposes, either to serve as food (e.g. meat) or to produce a product that serves as food (milk, eggs, etc.) or some other purpose, e.g. animals that are used for breeding, as companions or hobby pets, as show animals, for work or entertainment (e.g. sports), etc. The non-human subject may be domestic or wild, and/or may be located in a protected area such as a sanctuary or a zoo. Examples of suitable fish species that may be treated as described herein include but are not limited to: shellfish (including clams, crab, lobster, oysters, scallops and shrimp), trout and other stocking and food fish such as catfish, trout, salmon, walleyes, tilapia, bass, carp, bluegills, sunfish, perch, and eel. Examples of suitable avian species that may be treated as described herein include but are not limited to: chickens, ducks, geese, turkeys, guinea fowl, ostriches, pigeons, quails, and pheasants. Examples of suitable non-human mammals include but are not limited to: pigs, cattle, horses, sheep, goats, mice, rats, rabbits, guinea pigs, llamas, alpaca, addax, bison, camel, deer, donkey, eland, elk, gayal, mule, moose, oryx, water buffalo, yak, and zebu. In some aspects, the subject is a human.

The amount or dose of the combinations that is administered varies based on several factors, as will be understood by those of skill in the art. For example, the dose and frequency of administration varies according to the composition that is administered, the gender, age, weight, general physical condition, genetic background, etc. of the recipient. Generally, the dose will be in the range of from about 0.01 to about 1000 mg/kg of body weight or feed per day (e.g., about 0.1, 0.5, 1.0, 10, 20, 30, 40, 50, 60, 70, 80, 90, 100, 125, 150, 175, 200, 225, 250, 275, 300, 325, 350, 375, 400, 425, 450, 475 or 500 mg/kg, etc.).

The HDPs that are induced by administration of the compositions described herein include, but are not limited to: 14 avian β-defensins (AvBD1-14) and four cathelicidins (cath1-3 and cath-B1).

In some aspect, the combination of compounds is not: butyrate and acetate and propionate; butyrate and forskolin; butyrate and cholera toxin; butyrate and pertussis toxin; benzyl butyrate (BZB) and forskolin; glyceryl butyrate (GBT) and forskolin; butyrate and lactose; phenylbutyrate and lactose.

### EXAMPLES

### Example 1

### Induction of Host Defense Peptide Gene Expression by Short-Chain Fatty Acids, Histone Deacetylase Inhibitors, Cyclooxygenase-2 Inhibitors, Sugars, cAMP Signaling Agonists and Their Combinations

In this study, the HDP-inducing capacity of a group of structural and functional analogs of butyrate, medium- and long-chain fatty acids, mono- and disaccharide sugars, and COX-2 inhibitors was explored in chickens. In addition, their synergy with respect to HDP induction in vivo and/or in vitro was also investigated. Each compound was found to be capable of inducing HDPs in chickens, albeit with varying potency within and between each group. However, surprisingly, a strong synergy between butyrate (or its analogs) and sugars, cAMP signaling agonists, COX-2 inhibitors or other fatty acids in HDP induction was discovered. The results indicate that a combination of these compounds may be used as alternatives to antibiotics for growth promotion and disease control and prevention.

### Materials and Methods

### Chemicals

Sodium butyrate, lactose, galactose, dextrose, mannitol, trehalose, and sucrose were all obtained from Sigma-Aldrich (St. Louis, Mo.). Nimesulide, niflumic acid, resveratrol, anacardic acid, 2′-5′-dideoxyadenosine (DDA), PDTC, MG132, SB203580, PD98059, and SP600125 were procured from Santa Cruz Biotechnology (Santa Cruz, Calif.). Garcinol and quercetin were acquired from Cayman chemicals (Ann Arbor, Mich.).

### Cell Culture and RNA Isolation

Chicken HD11 macrophage cell line [12] was obtained from USDA-Agricultural Research Services (ARS), and chicken HTC macrophage cell line [13] was secured from USDA-ARS. Both HD11 and HTC macrophage cells were propagated in RPMI 1640 containing 10% fetal bovine serum (FBS), 100 μg/ml streptomycin, and 100 U/ml penicillin. Peripheral blood mononuclear cells (PBMCs) were isolated from EDTA-anticoagulated chicken blood by gradient centrifugation with Histopaque 1077 (Sigma) following manufacturer's instructions. Briefly, chicken blood was overlaid onto Histopaque 1077 (Sigma) at 1:1 ratio, and centrifuged at 400×g for 30 min. Interphase containing PBMCs was aspirated and washed 3 times with PBS at 250×g for 10 min. Cell pellet was re-suspended with RPMI 1640, supplemented with 10% FBS, 100 μg/ml streptomycin, 100 U/ml penicillin, and 20 mM HEPES. After being seeded overnight at 2×106/well in 6-well tissue culture plates, cells were treated with different concentrations of chemicals. For signaling studies, cells were pre-treated with different signaling agonists or inhibitors for 1 h, followed by stimulation with sodium butyrate or its structural analogs for another 24 h. Cells were then lysed in RNAzol (Molecular Research Center, Cincinnati, Ohio) for RNA extraction.

### Preparation, Culture, and Stimulation of Chicken Jejunal Explants

A segment of chicken jejunum was harvested from 1- to 2-week-old broiler chickens, washed thoroughly in cold PBS containing 100 μg/ml of gentamicin, 100 U/ml penicillin, and 100 μg/ml streptomycin, and then cut into a series of 0.5-cm-long segments. Jejunal segments were placed individually in 6-well plates and cultured in 4 ml RPMI 1640 containing 10% FBS, 20 mM HEPES, 100 U/ml penicillin, 100 μg/ml streptomycin, and 100 μg/ml gentamicin. Each segment was treated in triplicate with 4 mM butyrate with or without different concentrations of FSK, and then incubated in Hypoxia Chamber (StemCell Technologies, Vancouver, BC, Canada) filled with 95% O2 and 5% CO2 at 37° C. for 24 h. Jejunal segments were homogenized in RNAzol RT for RNA extraction.

### Real-Time RT-PCR

Total RNA was extracted from chicken cells and tissues using RNAzol RT (Molecular Research Center) and quantitated using Nanodrop 1000 (Nanodrop Products, Wilmington, Del.). RNA (0.3 μg) was reverse transcribed in 4-μl reactions using Maxima First Strand cDNA Synthesis Kit (Thermo Fisher Scientific, Pittsburgh, Pa.) according to the manufacturer's instructions. The cDNA was then diluted 10 times with RNase-free water prior to real-time PCR analysis of AvBD9 and GAPDH as were described previously [8, 14]. The primers used for AvBD9 primers were GCAAAGGCTATTCCACAGCAG (SEQ ID NO: 1, forward) and AGCATTTCAGTTCCCACCAC (SEQ ID NO: 2, reverse), whereas the primers for chicken glyceraldehyde-3-phosphate dehydrogenase (GAPDH) were GCACGCCATCACTATCTTCC (SEQ ID NO: 3, forward) and CATCCACCGTCTTCTGTGTG (SEQ ID NO: 4, reverse). QuantiTect SYBR Green PCR Kit (Qiagen, Valencia, Calif.) was used in 10 μl reactions with 4 and 1 μl of diluted cDNA for AvBD9 and GAPDH, respectively. Real-time PCR was performed with an initial activation at 95° C. for 10 min, followed by 40 cycles at 94° C. for 15 sec, 55° C. for 20 sec and 72° C. for 30 sec. Melting curve analysis was carried out to confirm the specificity of PCR amplification. Relative fold change in the AvBD9 gene expression was calculated using the ΔΔCt method normalized against the GAPDH levels.

### Immunoblotting

After stimulation with various compounds, chicken HD11 cells were washed with PBS and lysed in the RIPA lysis buffer (Santa Cruz Biotechnology, Dallas, Tex.). Protein concentration was measured by the Bradford Assay (Bio-Rad, Hercules, Calif.). For the determination of histone 4 acetylation levels, 20 μg of proteins was separated by 12.5% SDS-PAGE gel and then transferred to a PVDF membrane. After overnight blocking in the blocking buffer at 4° C., the membrane was incubated with a primary rabbit antibody against acetyl-histone 4 (Ac-H4) (Cell Signaling, #8647) in the blocking buffer for 1 h at room temperature. After three washes, the membrane was incubated with an alkaline phosphatase-conjugated goat anti-rabbit IgG antibody (Sigma-Aldrich, St. Louis, Mo.) for 45 min at room temperature. Another membrane was prepared in parallel, but incubated with a rabbit antibody against β-Actin (#A2103) to reveal a similar amount of protein loading. The signaling was visualized by incubation with the ECL reagent (Santa Cruz Biotechnology).

### Statistical Analysis

The results were expressed as mean±standard error from 2-3 independent experiments. Unpaired Student's two-tailed t-test was used for statistical evaluation using GraphPad Prism 5 (GraphPad Software, La Jolla, Calif.). The results were considered statistically significant if P<0.05.

### Results and Discussion

### Induction of HDP Gene Expression in Chicken Cells by Structural and Functional Analogs of Butyrate

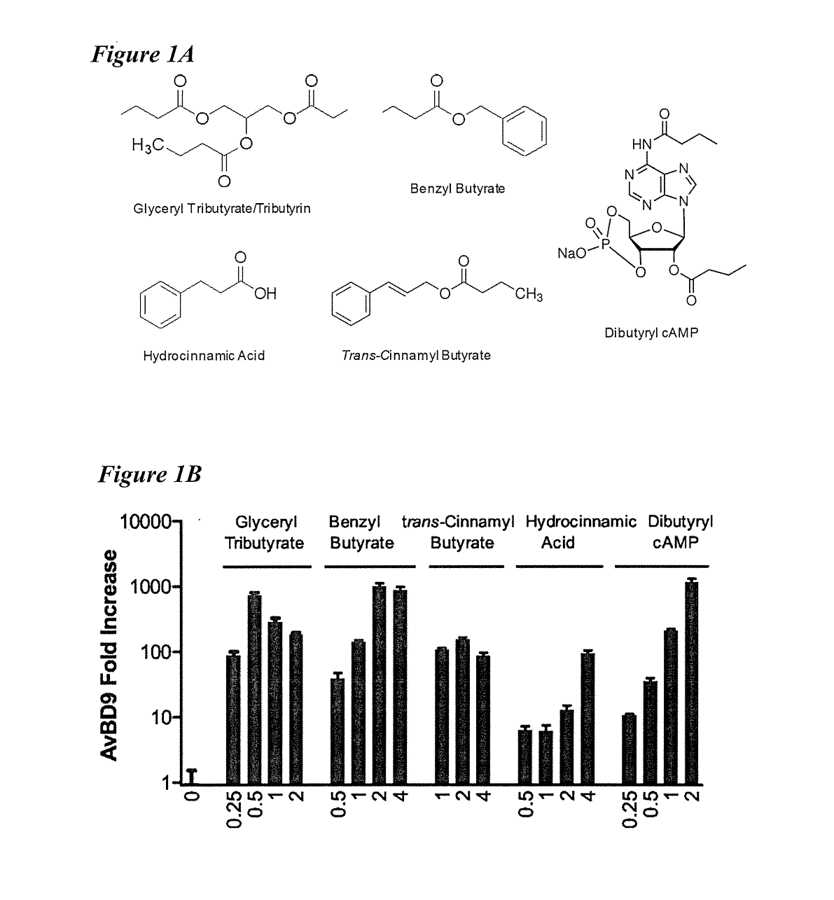

Although it is the most potent fatty acid in inducing HDP expression in chicken and porcine cells [8, 9], sodium butyrate is not particularly desirable for in vivo applications due to an unpleasant odor and quick metabolism in the intestinal tract [15]. Thus, several structural analogs of butyrate (FIG. 1A) were examined for their capacity to stimulate HDP synthesis. Glyceryl tributyrate (commonly known as tributyrin) is an odorless esterified butyrate analog consisting of three butyric acids with more favorable pharmacokinetic properties than butyrate [16]. Benzyl butyrate, on the other hand, is a butyrate derivative with a benzyl group linked to the carboxyl group of butyrate, and is a commonly used ingredient in fragrances with a pleasant plum smell [17]. Hydrocinnamic acid (also known as phenylpropanoic acid) is a propionate analog with a sweet, floral scent widely used in cosmetics, food additives, and pharmaceuticals [18]. On the other hand, trans-cinnamyl butyrate and dibutyryl cAMP are butyrate analogs (See FIG. 1A for their structures) that have been commonly used as fragrances and therapeutic drugs, respectively [19, 20].

Chicken β-defensin 9, as known as AvBD9, was chosen for gene expression studies because it is the most responsive gene to butyrate among all 14 chicken β-defensins and 4 cathelicidins [14]. Glyceryl tributyrate, benzyl butyrate, hydrocinnamic acid, trans-cinnamyl butyrate, and dibutyryl cAMP all dose-dependently induced AvBD9 gene expression in chicken HD11 cells following 24 h stimulation, with glyceryl tributyrate, benzyl butyrate, and dibutyryl cAMP achieving a comparable efficacy to butyrate (FIG. 1B). Because of their pleasant smell and/or structural rigidity, these butyrate analogs are expected to be more desirable than butyrate for applications in humans and live animals. The results are also consistent with our earlier findings in porcine [9] and human cells [10], where these butyrate analogs showed a similar HDP-inducing activity to butyrate, implying these analogs work in a species-independent manner.

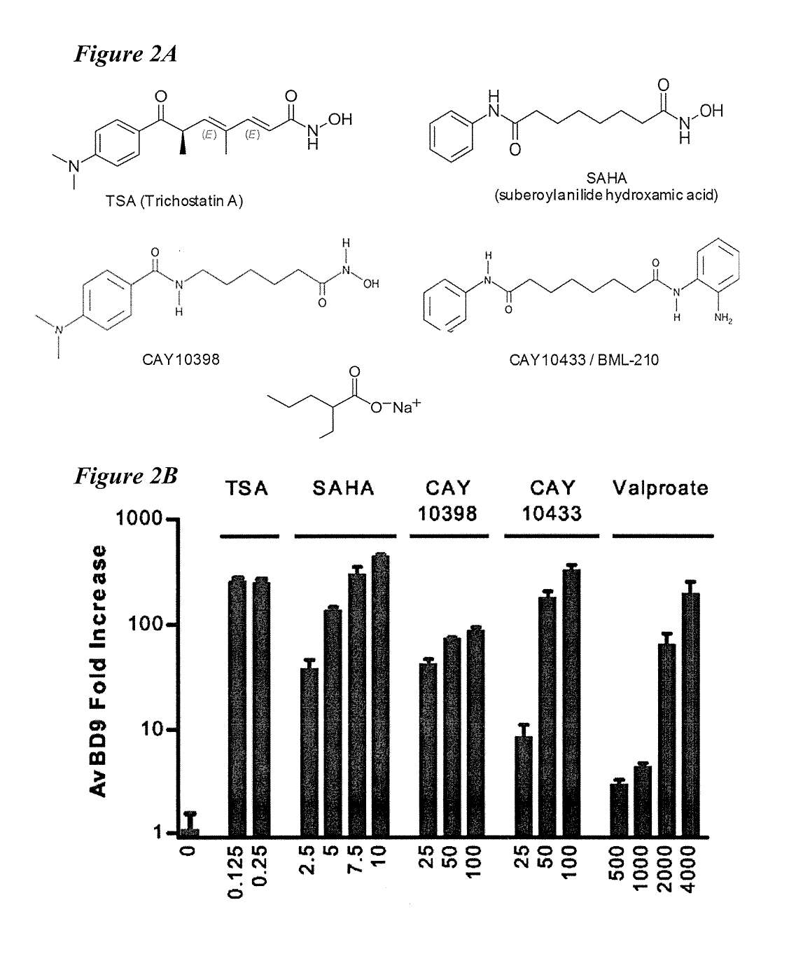

Because butyrate is also a well-known histone deacetylase inhibitor [21], the ability of its functional analogs were next evaluated, i.e., a panel of histone deacetylase inhibitors (FIG. 2A), in HDP gene induction. Indeed, trichistatin A, suberoylanilide hydroxamic acid (SAHA), sodium valproate, CAY10433/BML-210, and CAY10398 all showed a dose-dependent stimulation of AvBD9 gene expression in chicken HD11 cells (FIG. 2B). These results collectively suggested that, similar to butyrate, histone deacetylase inhibitors are excellent candidates for boosting host immunity, albeit with varying efficiencies. As SAHA and sodium valproate have been approved for various clinical manifestations [22], they can be included in feeds or foods for HDP induction and immune enhancement.

### Induction of HDP Gene Expression by Quercetin, a COX-2 Inhibitor

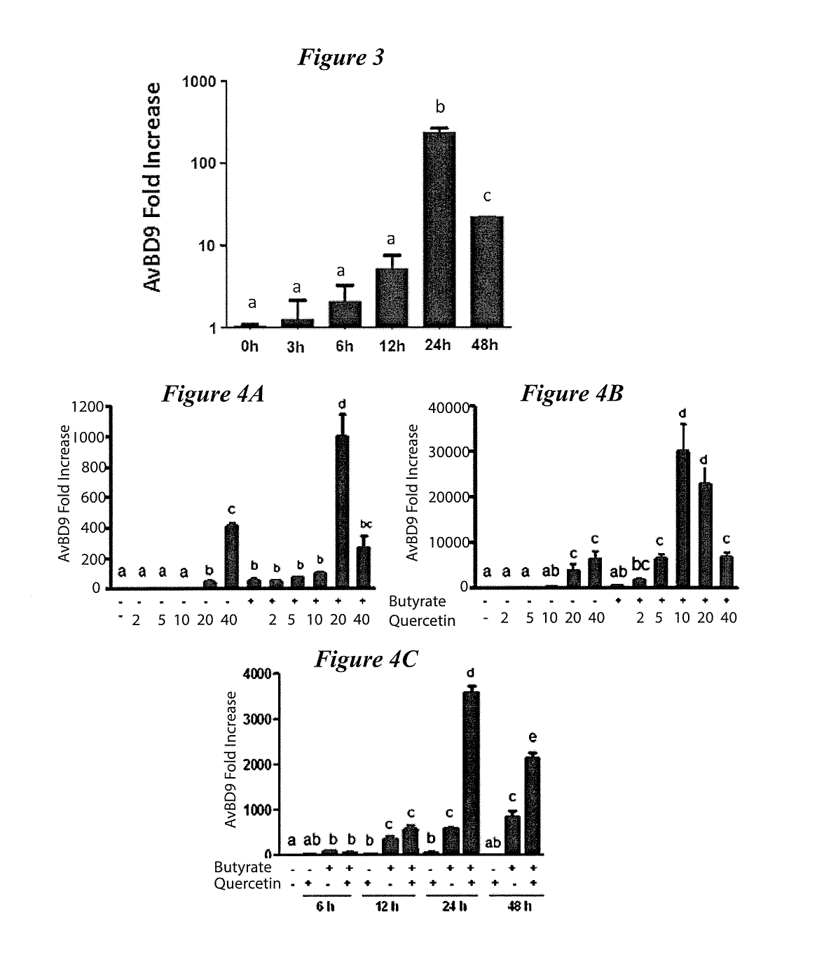

Quercetin is a flavonol and well-known COX-2 inhibitor that is naturally present in capers, cilantro, and red onions [23, 24]. To study the possible regulation of chicken AvBD9 gene expression by quercetin, chicken HTC macrophage cells were incubated with 40 μM quercetin for 0, 3, 6, 12, 24, or 48 h, followed by analysis of the AvBD9 gene expression by real-time RT-PCR. A time-dependent increase of AvBD9 expression was evident, peaking at 24 h with an approximately 500-fold increase (FIG. 3). However, the AvBD9 induction was reduced by 210-fold at 48 h, relative to 24 h, implying involvement of a negative feedback mechanism. An obvious dose-dependent induction of AvBD9 gene expression was also observed in HTC cells in response to quercetin, with 40 μM giving a peak response (FIG. 4A). Similarly, AvBD9 was also dose-dependently induced in chicken primary peripheral blood mononuclear cells (PBMCs) treated with quercetin, with 40 μM leading to an approximately 6,500-fold increase (FIG. 4B).

### Synergistic Augmentation of HDP Expression by a Combination of Butyrate and COX-2 Inhibitors

To explore a possible synergy in HDP induction between quercetin and butyrate, chicken HTC cells were treated with 2 mM butyrate in the presence or absence of different concentrations of quercetin for 24 h. An obvious dose-dependent synergistic induction of AvBD9 expression was seen between butyrate and quercetin, with the highest synergy seen with 20 μM quercetin and 2 mM butyrate (FIG. 4A). Similarly, a dose-dependent synergistic increase in AvBD9 gene expression was also observed in chicken PBMCs, with an approximately 3,000-fold induction occurring with 10 μM quercetin and 2 mM butyrate (FIG. 4B). Surprisingly, higher concentrations of quercetin, when combined with butyrate, led to a reduced synergy in both HTC cells and PBMCs (FIGS. 4A and 4B). To ensure 24-h stimulation giving the most robust synergy, a time-course experiment was done with PBMCs. Mimicking what was seen in HTC cells, 24-h incubation with a combination of butyrate and quercetin resulted in the highest synergistic induction with a nearly 4,000-fold increase in AvBD9 expression, followed by a slight decrease at 48 h (FIG. 4C).

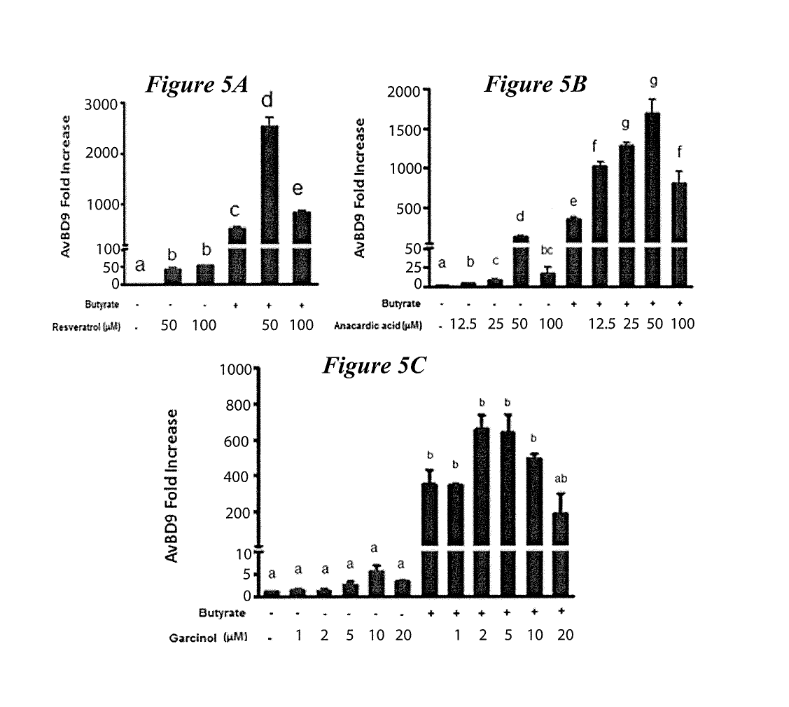

To further confirm the synergistic effect of butyrate combined with other natural COX-2 inhibitors on chicken HDP expression, chicken PBMCs were treated with different concentrations of resveratrol, anacardic acid and garcinol, followed by butyrate treatment for another 24 h. Treatment of PBMCs with resveratrol (FIG. 5A) or anacardic acid (FIG. 5B) alone had a modest effect on AvBD9 induction, with garcinol having the lowest induction of AvBD9 peaking at an approximately 7-fold increase (FIG. 5C). In contrast, a combination of 50 μM resveratrol (FIG. 5A) or 50 μM anacardic acid (FIG. 5B) with butyrate resulted in a strong synergistic induction of AvBD9 with a maximum fold change of approximately 2,500 and 1,500, respectively. When 2 mM butyrate was combined with 2 or 5 μM garcinol, an approximately 700-fold induction in AvBD9 expression was observed (FIG. 5C).

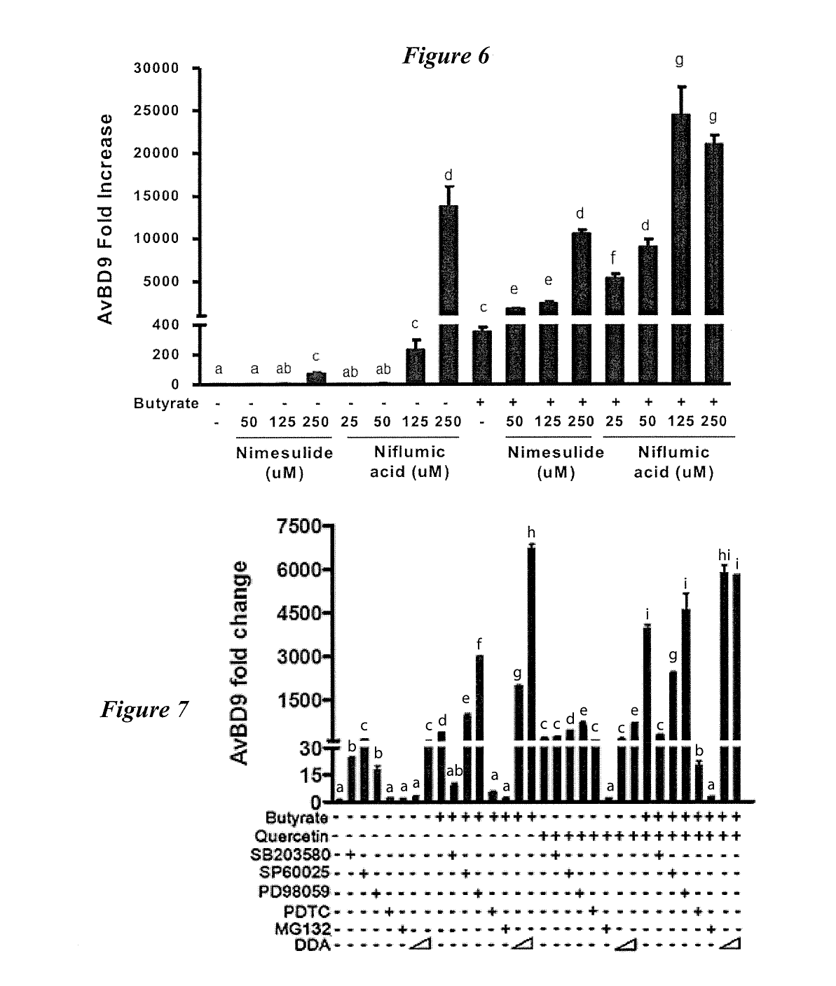

Nimesulide and niflumic acid are specific COX-2 inhibitors that inhibit the synthesis of PGE2 [25, 26]. To confirm the effect of specific COX-2 inhibition on induction of AvBD9 gene expression, PBMCs were stimulated with different concentrations of either nimesulide or niflumic acid alone or in combination with butyrate. Real-time RT-PCR revealed that both nimesulide and niflumic acid increased AvBD9 gene expression in a dose-dependent manner from 50 to 250 μM with a maximum fold increase of approximately 70 with nimesulide and 14,000 with niflumic acid (FIG. 6). More strikingly, co-stimulation of PBMCs with 250 μM nimesulide and 2 mM butyrate further enhanced AvBD9 gene expression with a 10,000-fold increase while a combination of 125 μM niflumic acid and butyrate resulted in an approximately 25,000-fold increase (FIG. 6). This further confirmed that inhibition of the COX-2 pathway is able to synergize with butyrate in up-regulating AvBD9 gene expression in chicken PBMCs.

Role of MAPK, NF-κB, and cAMP Signaling Pathways in AvBD9 Expression Induced by Butyrate and Quercetin

Three classical mitogen-activated protein kinase (MAPK) pathways, namely MEK-ERK, JNK, and p38 MAPK pathways, are important in the induction of a human HDP (LL-37) in response to butyrate, whereas the NF-κB pathway is dispensable in butyrate-mediated LL-37 expression [10]. To examine the role of MAPKs in butyrate- and quercetin-mediated AvBD9 induction, chicken PBMCs were treated with quercetin and butyrate in the presence or absence of a specific inhibitor for each of the three major MAPK pathways. To ensure the inhibitors themselves had a minimum impact on AvBD9 gene expression, PBMCs were treated with inhibitors alone. As expected, two NF-κB inhibitors did not influence AvBD9 expression. Somewhat surprisingly, inhibition of MAPKs and cAMP pathways resulted in an obvious, but modest AvBD9 induction in resting PBMCs (FIG. 7), suggesting that blocking MAPK and cAMP signaling is beneficial for constitutive AvBD9 expression in resting cells.

Blockage of the p38 MAPK pathway by SB203580 gave a substantial, but not complete reduction in AvBD9 expression in response to butyrate (FIG. 7), suggesting that p38 MAPK is partially required for butyrate-mediated AvBD9 induction. However, when butyrate was combined with the inhibitors to the JNK (SP600125) or MEK-ERK pathway (PD98059), a strong synergistic AvBD9 induction was observed (FIG. 7), suggesting that suppressing these two MAPK pathways is beneficial for butyrate-mediated HDP induction. A nearly complete abolishment of butyrate-mediated AvBD9 gene expression was seen when NF-κB was blocked by a specific inhibitor, MG132 (FIG. 7), indicative of a critical role of NF-κB in HDP expression. Inhibition of the cAMP signaling pathway by 5′-dideoxyadenosine (DDA), a cAMP signaling antagonist, showed a strikingly strong, dose-dependent increase in butyrate-induced AvBD9 expression (FIG. 7), implying that inhibiting cAMP signaling is synergistic with butyrate in AvBD9 induction.

In the case of quercetin, the MAPK and cAMP pathways appeared not to be involved in quercetin-meditated AvBD9 gene expression, as blocking either pathway had a minimal impact (FIG. 7). On the other hand, blocking NF-κB completely abolished quercetin-mediated AvBD9 expression, again alluding to the critical involvement of NF-κB. The reason that the other NF-κB inhibitor, PDTC, did not show much effect is unclear.

In the presence of MAPK, cAMP, and NF-κB inhibitors, cells responded to a combination of quercetin and butyrate similarly to butyrate alone. Inhibition of the MEK-ERK, INK, and cAMP pathways had a minimal impact on AvBD9 expression, whereas p38 MAPK was partially involved. Again, inhibition of NF-κB with both inhibitors substantially reduced AvBD9 gene induction in response to butyrate and quercetin, indicative of the necessity of NF-κB in AvBD9 expression.

### Induction of HDP Gene Expression by Sugars

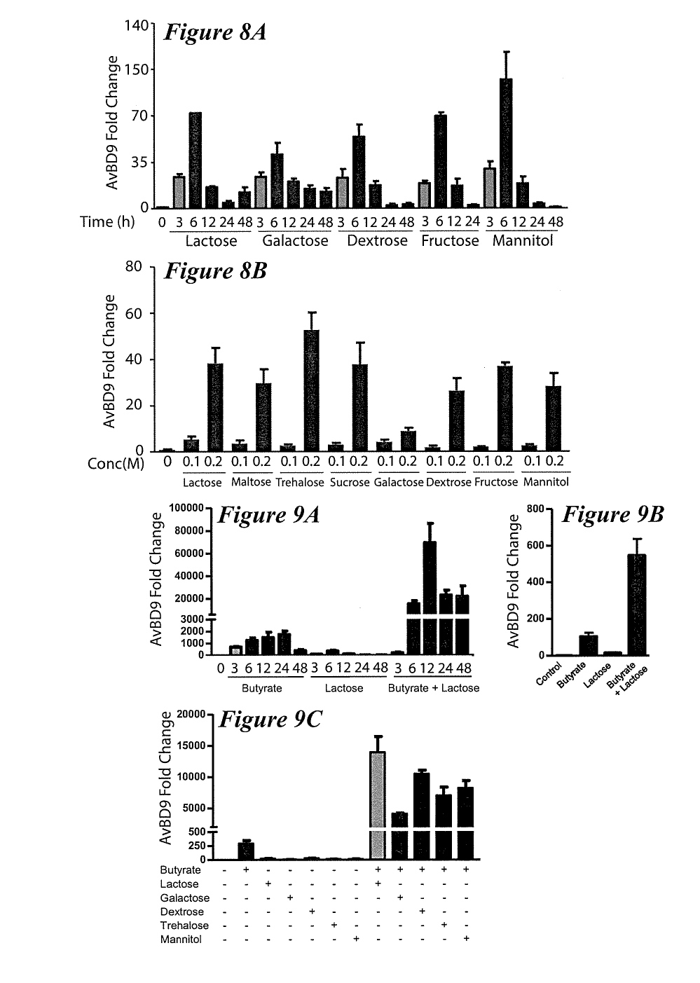

Several mono- and disaccharide sugars have been shown to induce HDP gene expression in human cells [6]. To determine whether chicken HDP genes can also be induced by sugars, chicken HD11 macrophages were treated with 0.2 M lactose, galactose, dextrose, and fructose for up to 48 h. All sugars clearly stimulated AvBD9 gene expression in a time-dependent manner. AvBD9 was readily induced as early as 3 h, peaked at 6 h, and graduate declined to nearly basal levels at 24 and 48 h (FIG. 8A). Sugar-mediated AvBD9 induction was also evidently dose-dependent. All sugars at 0.2 M gave a peak induction, whereas 0.1 M only showed a marginal effect at 6 h (FIG. 8B). Interestingly, mannitol, a sugar alcohol, also exhibited a similar time- and dose-dependent induction of AvBD9 (FIG. 8).

### Synergistic Induction of HDP Expression by a Combination of Butyrate and Sugars

To further explore whether sugars can synergize with butyrate in HDP induction, chicken HD11 cells were treated with 2 mM butyrate, 0.2 M lactose or a combination of both for 3, 6, 12, 24, and 48. As expected, butyrate triggered a peak response of an approximately 1,750-fold AvBD9 induction at 24 h, whereas lactose gave a maximum 350-fold AvBD9 induction at 6 h (FIG. 9A). Desirably, a combination of butyrate and lactose resulted in a synergistic

Induction of AvBD9 at all time points except for 3 h. A peak response was seen at 12 h with a nearly 70,000-fold increase in AvBD9 expression and the induction was sustained for at least 48 h (FIG. 9A). Besides HD11 cells, an obvious synergy was also observed between 2 mM butyrate and 0.1 M lactose in jejunal explants, with an approximately 550-fold AvBD9 induction in response to both agents for 24 h, whereas butyrate and lactose alone gave only 100-, and 15-fold increase in AvBD9 expression, respectively (FIG. 9B).

To confirm whether other mono- and disaccharides could also synergize with butyrate in inducing HDP expression, chicken HD11 cells were incubated with 2 mM butyrate or 0.2 M of different sugars individually or in combinations for 12 h. Similar to lactose, all other sugars such as galactose, dextrose and trehalose showed a clear synergy with butyrate in AvBD9 induction (FIG. 9C). Interestingly, mannitol also exhibited a comparably synergy with butyrate in inducing AvBD9 expression in HD11 cells (FIG. 9C).

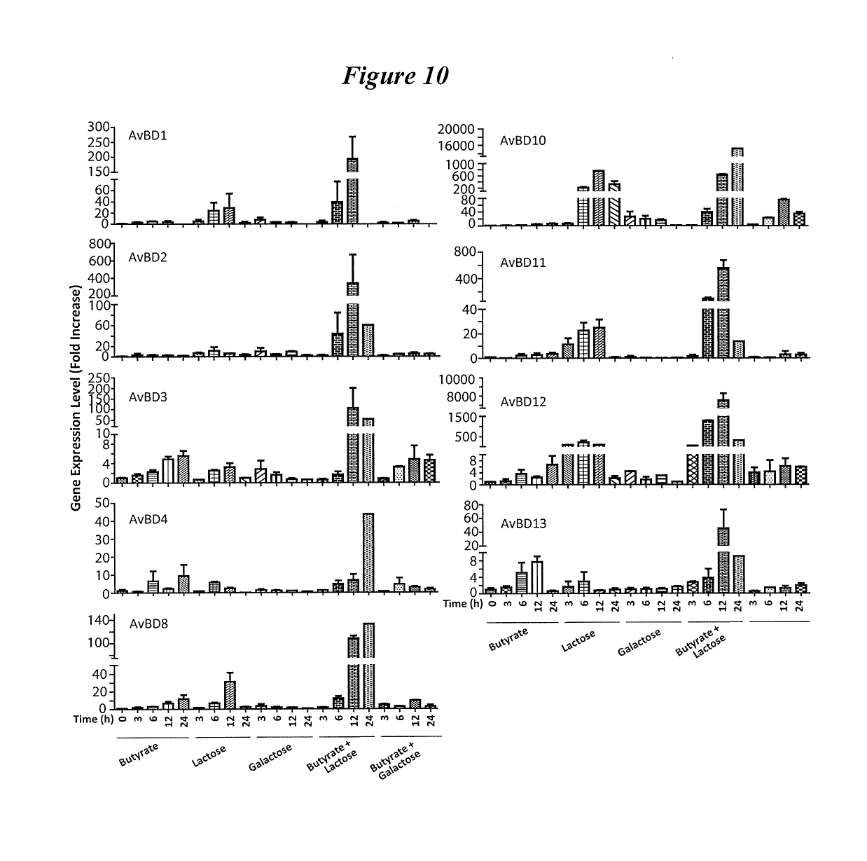

It is noted that, besides AvBD9, a majority of other chicken HDPs such as AvBD1, AvBD2, AvBD3, AvBD4, AvBD8, AvBD10, AvBD11, AvBD12, and AvBD13 were also synergistically induced by 2 mM butyrate and 0.2 M lactose at most time points, whereas the synergy between butyrate and galactose was more moderate in most cases (FIG. 10).

### Role of Histone Acetylation in AvBD9 Gene Expression Induced by Butyrate and Lactose

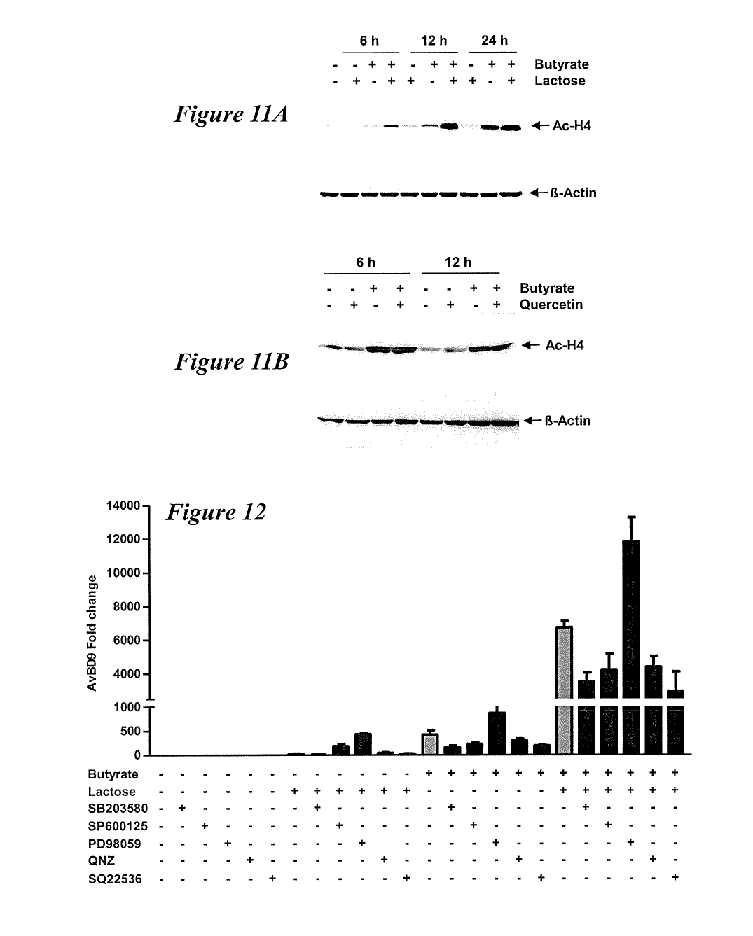

Butyrate induces HDP expression in chickens and humans mainly by acting as a histone deacetylase inhibitor (HDACi) [21]. To verify the impact of butyrate/lactose co-treatment on histone acetylation, chicken HD11 cells were treated with 2 mM butyrate and/or 0.2 M lactose for 6, 12, and 24 h, followed by evaluation of the acetylation status of histone 4 using immunoblotting. As shown in FIG. 11A, lactose alone had no impact on histone acetylation, and butyrate alone triggered minimum histone 4 acetylation at 6 h, but co-treatment with both agents caused an obvious acetylation of histone 4. Hyper-acetylation of histone 4 was also sustained at 12 h by a combination of butyrate and lactose, suggesting that enhanced histone acetylation is at least partially responsible for synergistic induction of AvBD9 expression by butyrate and lactose.

It is noted that butyrate/lactose-induced histone 4 hyper-acetylation was not obvious in comparison with butyrate alone (FIG. 11A), which is consistent with a diminished synergy in AvBD9 induction in HD11 cells treated with both butyrate and lactose at 24 and 48 h, relative to 12 h (FIG. 9A). In contrast to hyper-acetylation of histones induced by butyrate and lactose, combined treatment of HD11 cells with butyrate and quercetin failed to further augment histone acetylation in comparison with butyrate alone (FIG. 11B), implying that, unlike the butyrate/lactose synergy, the butyrate/quercetin synergy is caused by the reasons other than histone hyper-acetylation.

Role of MAPK, NF-κB, and cAMP Signaling Pathways in AvBD9 Induction Mediated by Butyrate and Lactose

MAPK, NF-κB, and cAMP signaling pathways are all involved in AvBD9 induction by butyrate and quercetin (FIG. 7). To examine the impact of these three important pathways on butyrate/lactose-mediated AvBD9 induction, chicken HD11 cells were treated for 24 h with butyrate and/or lactose in the presence or absence of specific inhibitors for each pathway. All inhibitors alone had a minimum influence on AvBD9 gene expression in HD11 cells as expected, whereas inhibition of p38 MAPK (by SB203580), NF-κB (by QNZ), and cAMP pathway (by SQ22536) had no influence on lactose-induced AvBD9 expression, suggestive of no involvement of these pathways (FIG. 12). Surprisingly, blocking the JNK pathway by SP600125 or the MEK-ERK pathway by PD98059 synergized substantially with lactose in inducing AvBD9 in HD11 cells (FIG. 12).

On the other hand, inhibition of p38 MAPK, INK, NF-κB, and cAMP pathways partially suppressed butyrate-induced AvBD9 expression, suggesting that these pathways are partially involved (FIG. 12). Blockage of the MEK-ERK pathway also enhanced butyrate-triggered AvBD9 expression (FIG. 12), implying that inhibiting the MEK-ERK would synergize with butyrate in AvBD9 induction. The role of these pathways in the butyrate-lactose synergy is similar to that in the butyrate alone. The p38 MAPK, JNK, NF-κB, and cAMP pathways were partially required for AvBD9 induction by a combination of butyrate and lactose, whereas inhibiting the MEK-ERK pathway potentiated the butyrate-lactose synergy in chicken HD11 cells.

Synergistic Induction of HDP Expression with a Combination of Butyrate, Forskolin, and Lactose

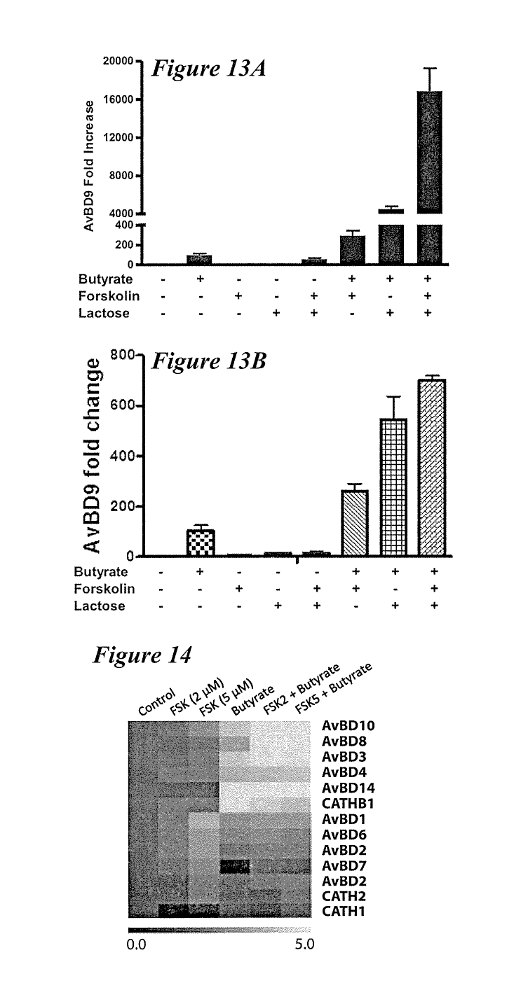

To examine a possible additional synergy among three different compounds, chicken HD11 cells were treated with butyrate, forskolin, and lactose individually or in combinations of 2-3 for 24 h, followed by real-time RT-PCR analysis of AvBD9 expression levels. As expected, when butyrate was combined with forskolin or lactose, an obvious synergy in AvBD9 induction was observed (FIG. 13A). However, a minimum synergy exhibited between forskolin and lactose (FIG. 13A). Desirably, a drastic synergistic interaction in augmenting AvBD9 expression was seen with a combination of butyrate, forskolin and lactose, with approximately additional 4- and 55-fold increases in AvBD9 expression, relative to butyrate/lactose and butyrate/forskolin, respectively (FIG. 13A). A similar trend also occurred in chicken jejunal explants that were treated with butyrate, forskolin, and lactose individually or in combinations. The strongest synergy was observed when all three compounds were combined (FIG. 13B). Besides AvBD9, several other chicken HDP genes such as AvBD3, AvBD4, AvBD8, and AvBD10 were also synergistically induced by a combination of butyrate and forskolin in HD11 cells, whereas the remaining HDPs were minimally affected (FIG. 14).

### Synergistic Induction of HDP Expression Among Fatty Acids

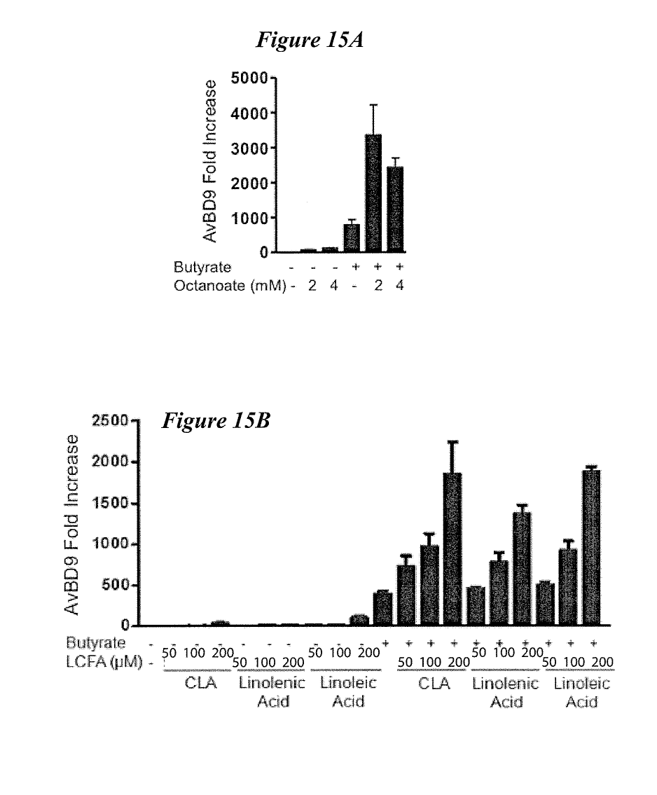

Short-chain fatty acids including butyrate, propionate, and acetate synergize with each other in inducing chicken HDP expression in both cells and live animals [8]. When butyrate was combined with a medium-chain fatty acid, namely sodium octanoate, a synergy in AvBD9 induction was evident in chicken HD11 cells, with an additional 4-fold increase relative to butyrate alone (FIG. 15A). A similar synergy in AvBD9 induction was also seen between butyrate and any of the three long-chain fatty acids including conjugated linoleic acids (CLA), linolenic acid, and linoleic acid. Although long-chain fatty acids alone had a marginal effect on AvBD9 induction, they triggered an obvious further increase in AvDB9 expression when combined with butyrate (FIG. 15B).

An aspect of the invention disclosed herein is the synergy exhibited between various classes of compounds when used to enhance the synthesis of endogenous host defense peptides. For example, FIGS. 4, 5, and 6 demonstrated the synergy between butyrate (a Class I compound) and several different natural and synthetic COX-2 inhibitors (Class II compounds), whereas a strong synergy between butyrate (a Class I compound) and sugars (Class III compounds) was shown in FIG. 9. A combination of three compounds, with one in Class I (butyrate), one in Class III [lactose (a sugar)] and one in Class IV [forskolin (a cAMP agonist)] was also indicated in FIG. 13. In FIG. 15, the synergy between butyrate and its structural analogs, all within Class I, is shown.

In summary, short-chain fatty acids, and butyrate in particular, are strong inducers of HDP gene expression in chickens and pigs. Structural analogs of butyrate such as, by way of example, glyceryl tributyrate, benzyl butyrate, trans-cinnamyl butyrate, and hydrocinnamic acid are all capable of inducing HDP expression in chickens and pigs. At the same time, functional analogs of butyrate, i.e., histone deacetylase inhibitors such as sodium valproate and suberoylanilide hydroxamic acid (SAHA), have a strong capacity to induce HDP expression in chickens. Mono- and disaccharide sugars such as galactose, dextrose, lactose, maltose, sucrose, and trehalose are all capable of inducing HDP expression in chickens. Additionally, agonists of the cAMP signaling pathway such as forskolin are able to stimulate HDP expression in chickens. Furthermore, COX-2 inhibitors such as quercetin, resveratrol, garcinol, nimesulide and niflumic acid are also strong inducers of HDP expression in chickens. Desirably, in an embodiment a combination of sodium butyrate with a sugar, a cAMP agonist, a cyclooxygenase-2 inhibitor, and/or a fatty acid (short-, medium- or long-chain) are synergistic in inducing HDP expression in chickens. Collectively, these results suggested that butyrate analogs, fatty acids, histone deacetylase inhibitors, sugars, cAMP signaling agonists, COX-2 inhibitors and their combinations with a strong capacity to induce HDP synthesis may have potential to augment animal innate immunity and be developed as effective alternatives to antibiotics for use in livestock and companion animals. Because of the conservation of this innate immune mechanism, the results are potentially applicable to humans.

The combinations of a compound in Class I or Class II with 1-2 compounds in Classes III, IV or V have been shown in many cases to synergize with each other in inducing host defense peptide synthesis. Furthermore, several compounds in Class I also synergize with each other (FIG. 15).

The relative amounts of individual compounds in each combination can potentially vary greatly because of a huge variation in the relative potency of each compound, will need to be determined empirically. Those of ordinary skill in the art will readily understand how to determine effective relative proportions of different classes of compounds in a particular case, i.e., for a given combination and subject animal, where animal includes humans.

In some embodiments, butyrate and many other fatty acids can be effective in humans cells at similar concentrations to those used in the chicken and porcine cells (PLoS One 2012, 7: e49558; PLoS One 2013, 8: e72922; Peptides 2013, 50: 129-138). One embodiment will utilize whatever concentrations/combinations that work in chickens and/or pigs in applications for humans and other animals.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such teiins should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100, such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

### REFERENCES

1. Zasloff, M. (2002) Antimicrobial peptides of multicellular organisms, Nature. 415, 389-95.
2. Brogden, K. A., Ackermann, M., McCray, P. B., Jr. & Tack, B. F. (2003) Antimicrobial peptides in animals and their role in host defences, International journal of antimicrobial agents. 22, 465-78.
3. Hancock, R. E. & Sahl, H. G. (2006) Antimicrobial and host-defense peptides as new anti-infective therapeutic strategies, Nat Biotechnol. 24, 1551-7.
4. Yang, D., Biragyn, A., Hoover, D. M., Lubkowski, J. & Oppenheim, J. J. (2004) Multiple roles of antimicrobial defensins, cathelicidins, and eosinophil-derived neurotoxin in host defense, Annual review of immunology. 22, 181-215.
5. Schauber, J., Svanholm, C., Termen, S., Iffland, K., Menzel, T., Scheppach, W., Melcher, R., Agerberth, B., Luhrs, H. & Gudmundsson, G. H. (2003) Expression of the cathelicidin LL-37 is modulated by short chain fatty acids in colonocytes: relevance of signalling pathways, Gut. 52, 735-41.
6. Cederlund, A., Kai-Larsen, Y., Printz, G., Yoshio, H., Alvelius, G., Lagercrantz, H., Stromberg, R., Jornvall, H., Gudmundsson, G. H. & Agerberth, B. (2013) Lactose in human breast milk an inducer of innate immunity with implications for a role in intestinal homeostasis, PloS one. 8, e53876.
7. Sunkara, L. T., Achanta, M., Schreiber, N. B., Bommineni, Y. R., Dai, G., Jiang, W., Lamont, S., Lillehoj, H. S., Beker, A., Teeter, R. G. & Zhang, G. (2011) Butyrate enhances disease resistance of chickens by inducing antimicrobial host defense peptide gene expression, PLoS One (In Press).
8. Sunkara, L. T., Jiang, W. & Zhang, G. (2012) Modulation of antimicrobial host defense peptide gene expression by free fatty acids, PloS one. 7, e49558.
9. Zeng, X., Sunkara, L. T., Jiang, W., Bible, M., Carter, S., Ma, X., Qiao, S. & Zhang, G. (2013) Induction of porcine host defense Peptide gene expression by short-chain Fatty acids and their analogs, PloS one. 8, e72922.
10. Jiang, W., Sunkara, L. T., Zeng, X., Deng, Z., Myers, S. M. & Zhang, G. (2013) Differential regulation of human cathelicidin LL-37 by free fatty acids and their analogs, Peptides. 50, 129-38.
11. Sunkara, L. T., Zeng, X., Curtis, A. R. & Zhang, G. (2014) Cyclic AMP synergizes with butyrate in promoting beta-defensin 9 expression in chickens, Molecular immunology. 57, 171-80.
12. Beug, H., von Kirchbach, A., Doderlein, G., Conscience, J. F. & Graf, T. (1979) Chicken hematopoietic cells transformed by seven strains of defective avian leukemia viruses display three distinct phenotypes of differentiation, Cell. 18, 375-90.
13. Rath, N. C., Parcells, M. S., Xie, H. & Santin, E. (2003) Characterization of a spontaneously transformed chicken mononuclear cell line, Veterinary immunology and immunopathology. 96, 93-104.
14. Sunkara, L. T., Achanta, M., Schreiber, N. B., Bommineni, Y. R., Dai, G., Jiang, W., Lamont, S., Lillehoj, H. S., Beker, A., Teeter, R. G. & Zhang, G. (2011) Butyrate enhances disease resistance of chickens by inducing antimicrobial host defense peptide gene expression, PloS one. 6, e27225.
15. van der Does, A. M., Bergman, P., Agerberth, B. & Lindbom, L. (2012) Induction of the human cathelicidin LL-37 as a novel treatment against bacterial infections, Journal of leukocyte biology. 92, 735-42.
16. Heidor, R., Ortega, J. F., de Conti, A., Ong, T. P. & Moreno, F. S. (2012) Anticarcinogenic actions of tributyrin, a butyric acid prodrug, Current drug targets. 13, 1720-9.
17. McGinty, D., Letizia, C. S. & Api, A. M. (2012) Fragrance material review on benzyl butyrate, Food and chemical toxicology: an international journal published for the British Industrial Biological Research Association. 50 Suppl 2, S407-11.
18. Korneev, S. M. (2013) Hydrocinnamic Acids: Application and Strategy of Synthesis, Synthesis-Stuttgart. 45, 1000-1015.
19. Bhatia, S. P., Wellington, G. A., Cocchiara, J., Lalko, J., Letizia, C. S. & Api, A. M. (2007) Fragrance material review on cinnamyl butyrate, Food Chem Toxicol. 45, S62-S65.
20. Schwede, F., Maronde, E., Genieser, H. G. & Jastorff, B. (2000) Cyclic nucleotide analogs as biochemical tools and prospective drugs, Pharmacol Therapeut. 87, 199-226.
21. Davie, J. R. (2003) Inhibition of histone deacetylase activity by butyrate, The Journal of nutrition. 133, 2485S-2493S.
22. Batty, N., Malouf, G. G. & Issa, J. P. (2009) Histone deacetylase inhibitors as anti-neoplastic agents, Cancer letters. 280, 192-200.
23. Xiao, X., Shi, D., Liu, L., Wang, J., Xie, X., Kang, T. & Deng, W. (2011) Quercetin suppresses cyclooxygenase-2 expression and angiogenesis through inactivation of P300 signaling, PloS one. 6, e22934.
24. Cheong, E., Ivory, K., Doleman, J., Parker, M. L., Rhodes, M. & Johnson, I. T. (2004) Synthetic and naturally occurring COX-2 inhibitors suppress proliferation in a human oesophageal adenocarcinoma cell line (OE33) by inducing apoptosis and cell cycle arrest, Carcinogenesis. 25, 1945-52.
25. Suleyman, H., Cadirci, E., Albayrak, A. & Halici, Z. (2008) Nimesulide is a selective COX-2 inhibitory, atypical non-steroidal anti-inflammatory drug, Current medicinal chemistry. 15, 278-83.
26. Kim, B. M., Maeng, K., Lee, K. H. & Hong, S. H. (2011) Combined treatment with the Cox-2 inhibitor niflumic acid and PPARgamma ligand ciglitazone induces ER stress/caspase-8-mediated apoptosis in human lung cancer cells, Cancer letters. 300, 134-44.

What is claimed is:

1. A composition comprising
i) a short-chain fatty acid or a chemical or functional analog thereof; and
ii) at least one compound that is
a different short-, medium- or long-chain fatty acid or chemical or functional analog thereof;
a monosaccharide or a disaccharide,
a cyclic adenosine monophosphate (cAMP) agonist, and/or
a cyclooxygenase-2 inhibitor,
with the caveat that the combination is not: butyrate and acetate and propionate, butyrate and forskolin, butyrate and cholera toxin, butyrate and pertussis toxin, benzyl butyrate and forskolin, glyceryl butyrate and forskolin, butyrate and lactose, or phenylbutyrate and lactose.

2. The composition of claim 1, wherein:
the short-chain fatty acid is selected from the group consisting of: acetic (C2), propionic (C3), butyric (C4), isobutyric acid, valeric acid (C5), isovaleric acid, and salts thereof;
the medium-chain fatty acid is selected from the group consisting of: caproic (C6), enanthic (C7), caprylic (C8), pelargonic (C9), capric (C10), undecylic (C11), and lauric acid (C12), and their isomers, and salts thereof; and
the saturated or unsaturated long-chain fatty acid is selected from the group consisting of: tridecylic (C13), myristic (C14), pentadecanoic (C15), palmitic (C16), margaric (C17), stearic (C18), nonadecylic (C19), arachidic (C20), heneicosylic (C21), behenic (C22), α-linolenic (18:3), stearidonic (18:4), eicosapentaenoic (20:5), docosahexaenoic (22:6), linoleic (18:2), conjugated linoleic, γ-linolenic (18:3), dihomo-γ-linolenic (20:3), arachidonic (20:4), adrenic (22:4), palmitoleic (16:1), vaccenic (18:1), paullinic (20:1), oleic (18:1), elaidic (trans-18:1), gondoic (20:1), erucic (22:1), nervonic (24:1), and mead acid (20:3), and their isomers, and salts thereof.

3. The composition of claim 1, wherein the chemical analog is a chemical analog of short-chain fatty acids.

4. The composition of claim 3, wherein the chemical analog of short-chain fatty acids is selected from the group including but not limited to: monoglyceride, diglyceride and triglyceride analogs of short-chain fatty acids such as glyceryl tributyrate, glyceryl dibutyrate, and glyceryl monobutyrate; benzyl analogs such as benzyl butyrate, benzyl propionate and benzyl valerate; cinnamyl and trans-cinnamyl analogs such as trans-cinnamylbutyrate; and short-chain fatty acids with a phenyl group attached such as hydrocinnamic acid and 4-phenylbutyrate; and salts thereof.

5. The composition of claim 1, wherein the functional analog is a functional analog of butyrate.

6. The composition of claim 5, wherein the functional analog of butyrate is a histone deacetylase inhibitor.

7. The composition of claim 6, wherein the histone deacetylase inhibitor includes, but is not limited to: sodium valproate, Vorinostat (SAHA, MK0683), trichistatin A, CAY10433/BML-210, CAY10398, Entinostat (MS-275), Chidamide, Trichostatin A (TSA), Panobinostat (LBH589), Mocetinostat (MGCD0103), Belinostat (PXD101), Romidepsin (FK228, Depsipeptide), MC1568, Tubastatin A-HCl, Tubastatin A, Givinostat (ITF2357), LAQ824 (Dacinostat), CUDC-101, Quisinostat (JNJ-26481585), Pracinostat (SB939), PCI-34051, Droxinostat, PCI-24781 (Abexinostat), RGFP966, AR-42, Rocilinostat (ACY-1215), CI994 (Tacedinaline), CUDC-907, M344, Tubacin, RG2833 (RGFP109), Resminostat, BRD73954, BG45, 4SC-202, CAY10603, LMK-235, Nexturastat A, TMP269, Scriptaid, and HPOB.

8. The composition of claim 1, wherein:
the monosaccharide is selected from the group including but not limited to: the D- and L-isomers of hexoses (allose, altrose, glucose, mannose, gulose, idose, galactose, talose, fructose, psicose, sorbose, and tagatose), and
the disaccharide is selected from the group including but not limited to: sucrose, lactose, maltose, trehalose, lactulose, and cellobiose.

9. The composition of claim 1, wherein the cyclic adenosine monophosphate (cAMP) agonist is selected from the group including, but not limited to: 8-bromo-cAMP, forskolin, cholera toxin (CT), pertussis toxin (PT), dibutyryl cAMP (or bucladesine), caffeine, and theophylline.

10. The composition of claim 1, wherein the cyclooxygenase-2 inhibitor is selected from the group including, but not limited to: quercetin, resveratrol, garcinol, anacardic acid, curcumin, epigallocatechin-3 galate, pycnogenol nimesulide, niflumic acid, celecoxib, etoricoxib, and rofecoxib.

11. The composition of claim 1, wherein the composition comprises: a combination of two compounds (e.g., butyrate, its chemical analog or a histone deacetylase inhibitor in combination with a cyclooxygenase-2 inhibitor such as quercetin, resveratrol, garcinol, anacardic acid, curcumin or epigallocatechin-3 galate), or a combination of three compounds (e.g., butyrate, its chemical analog or a histone deacetylase inhibitor in combination with a cAMP agonist such as forskolin and a sugar such as lactose).

12. A method of synergistically increasing a level of expression of one or more genes encoding a host defense peptide in a subject, comprising
administering to the subject a composition comprising
i) a short-chain fatty acid or a chemical or functional analog thereof; and
ii) at least one compound that is
a different short, medium, or long-chain fatty acid or chemical or functional analog thereof;
a monosaccharide or a disaccharide,
a cyclic adenosine monophosphate (cAMP) agonist and/or
a cyclooxygenase-2 inhibitor,
wherein the composition is administered in an amount sufficient to increase the level of expression of one or more genes encoding the host defense peptides,
with the caveat that the combination is not: butyrate and acetate and propionate, butyrate and forskolin, butyrate and cholera toxin, butyrate and pertussis toxin, benzyl butyrate and forskolin, glyceryl butyrate and forskolin, butyrate and lactose, or phenylbutyrate and lactose.

13. The method of claim 12, wherein:
the short-chain fatty acid is selected from the group consisting of: acetic (C2), propionic (C3), butyric (C4), isobutyric acid, valeric acid (C5), isovaleric acid, and salts thereof;
the medium-chain fatty acid is selected from the group consisting of: caproic (C6), enanthic (C7), caprylic (C8), pelargonic (C9), capric (C10), undecylic (C11), and lauric acid (C12), and their isomers, and salts thereof; and
the saturated or unsaturated long-chain fatty acid is selected from the group consisting of: tridecylic (C13), myristic (C14), pentadecanoic (C15), palmitic (C16), margaric (C17), stearic (C18), nonadecylic (C19), arachidic (C20), heneicosylic (C21), behenic (C22), α-linolenic (18:3), stearidonic (18:4), eicosapentaenoic (20:5), docosahexaenoic (22:6), linoleic (18:2), conjugated linoleic, γ-linolenic (18:3), dihomo-γ-linolenic (20:3), arachidonic (20:4), adrenic (22:4), palmitoleic (16:1), vaccenic (18:1), paullinic (20:1), oleic (18:1), elaidic (trans-18:1), gondoic (20:1), erucic (22:1), nervonic (24:1), and mead acid (20:3), and their isomers, and salts thereof.

14. The method of claim 12, wherein the chemical analog is a chemical analog of short-chain fatty acid.

15. The method of claim 14, wherein the chemical analog of short-chain fatty acids is selected from the group including but not limited to: monoglyceride, diglyceride and triglyceride analogs of short-chain fatty acids such as glyceryl tributyrate, glyceryl dibutyrate, and glyceryl monobutyrate; benzyl analogs such as benzyl butyrate, benzyl propionate and benzyl valerate; cinnamyl and trans-cinnamyl analogs such as trans-cinnamylbutyrate; and short-chain fatty acids with a phenyl group attached such as hydrocinnamic acid and 4-phenylbutyrate; and salts thereof.

16. The method of claim 12, wherein the functional analog is a functional analog of butyrate.

17. The method of claim 16, wherein the functional analog of butyrate is a histone deacetylase inhibitor.

18. The method of claim 17, wherein the histone deacetylase inhibitor includes, but is not limited to: sodium valproate, Vorinostat (SAHA, MK0683), trichistatin A, CAY10433/BML-210, CAY10398, Entinostat (MS-275), Chidamide, Trichostatin A (TSA), Panobinostat (LBH589), Mocetinostat (MGCD0103), Belinostat (PXD101), Romidepsin (FK228, Depsipeptide), MC1568, Tubastatin A-HCl, Tubastatin A, Givinostat (ITF2357), LAQ824 (Dacinostat), CUDC-101, Quisinostat (JNJ-26481585), Pracinostat (SB939), PCI-34051, Droxinostat, PCI-24781 (Abexinostat), RGFP966, AR-42, Rocilinostat (ACY-1215), CI994 (Tacedinaline), CUDC-907, M344, Tubacin, RG2833 (RGFP109), Resminostat, BRD73954, BG45, 4SC-202, CAY10603, LMK-235, Nexturastat A, TMP269, Scriptaid, and HPOB.

19. The method of claim 12, wherein:
the monosaccharide is selected from the group including but not limited to: the D- and L-isomers of hexoses (allose, altrose, glucose, mannose, gulose, idose, galactose, talose, fructose, psicose, sorbose, and tagatose), and
the disaccharide is selected from the group including but not limited to: sucrose, lactose, maltose, trehalose, lactulose, and cellobiose.

20. The method of claim 12, wherein the cyclic adenosine monophosphate (cAMP) agonist is selected from the group including, but not limited to: 8-bromo-cAMP, forskolin, cholera toxin (CT), pertussis toxin (PT), dibutyryl cAMP (or bucladesine), caffeine, and theophylline.

21. The method of claim 12, wherein the cyclooxygenase-2 inhibitor is selected from the group including, but not limited to: quercetin, resveratrol, garcinol, anacardic acid, curcumin, epigallocatechin-3 galate, pycnogenol nimesulide, niflumic acid, celecoxib, etoricoxib, and rofecoxib.

22. The method of claim 12, wherein the composition comprises: a combination of two compounds (e.g., butyrate, its chemical analog or a histone deacetylase inhibitor in combination with a cyclooxygenase-2 inhibitor such as quercetin, resveratrol, garcinol, anacardic acid, curcumin or epigallocatechin-3 galate), or a combination of three compounds (e.g., butyrate, its chemical analog or a histone deacetylase inhibitor in combination with a cAMP agonist such as forskolin and a sugar such as lactose).

23. The method of claim 12, wherein the one or more genes is/are selected from the group consisting of: 14 avian β-defensins (AvBD1-14) and four cathelicidins (cath1-3 and cath-B1).
